(19) United States
(12) Patent Application Publication
(10) Pub. No.: US 2025/0114351 A1
(43) Pub. Date: Apr. 10, 2025

(54) 5-HT2C RECEPTOR MODULATING AGENTS FOR THE TREATMENT OF NEURODEGENERATIVE, MENTAL, COGNITIVE AND AUTOIMMUNE CNS DISORDERS

(71) Applicant: Technion Research & Development Foundation Limited, Haifa (IL)

(72) Inventors: Asya ROLLS, Haifa (IL); Dorit FARFARA, Haifa (IL); Ben KORIN, Haifa (IL); Hilla AZULAY-DEBBY, Haifa (IL); Shahar ARMON, Haifa (IL)

(73) Assignee: Technion Research & Development Foundation Limited, Haifa (IL)

(21) Appl. No.: 18/985,200

(22) Filed: Dec. 18, 2024

Related U.S. Application Data
(63) Continuation of application No. PCT/IL2023/050641, filed on Jun. 21, 2023.
(60) Provisional application No. 63/353,873, filed on Jun. 21, 2022.

(51) Int. Cl. A61K 31/496; A61K 39/395

(52) U.S. Cl. CPC A61K 31/496; A61K 39/3955

(57) ABSTRACT

A method of treating a disease or disorder for which an increase in immune cells in the brain is therapeutic in a subject is disclosed. The method comprises administering to the subject a therapeutically effective amount of an agent which specifically binds and increases activity of a receptor selected from the group consisting of a 5HT2C receptor, a 5HT2A receptor and a 5HT2B receptor in the postcentral gyrus and/or the choroid plexus (CP) of the brain of the subject. Compositions for treating these diseases are also disclosed.

### RELATED APPLICATIONS

This application is a Continuation of PCT Patent Application No. PCT/IL2023/050641 having International filing date of Jun. 21, 2023, which claims the benefit of priority under 35 USC § 119(e) of U.S. Provisional Patent Application No. 63/353,873 filed on Jun. 21, 2022. The contents of the above applications are all incorporated by reference as if fully set forth herein in their entirety.

### FIELD AND BACKGROUND OF THE INVENTION

The present invention, in some embodiments thereof, relates to methods of treating neurodegenerative, mental, cognitive and autoimmune disorders using agent which specifically target the 5-HT_{2C} receptor.

The 5-HT_{2C} receptors are one of three subtypes that belong to the serotonin 5-HT₂ receptor subfamily along with 5-HT_{2A} and 5-HT_{2B} receptors.

The 5-HT_{2C} receptor is a G-protein coupled receptor. It is expressed almost exclusively in the central nervous system, including the hypothalamus, hippocampus, tonsillar nucleus, solitary tract nucleus, spinal cord, cortex, olfactory bulb, ventral tegmental area (ATV), nucleus accumbens and choroid plexus.

The development of 5-HT_{2C} agonists has been a major obstacle, because of severe side effects due to a lack of selectivity over 5-HT_{2A} and 5-HT_{2B} receptors. Activation of 5-HT_{2A} receptors can induce hallucinations, and the activation of 5-HT_{2B} receptors has been implicated in cardiac valvular insufficiency and possibly in pulmonary hypertension. 5-HT_{2C} receptors are widely distributed in the brain, and mediate regulatory effects of 5-HT on anxiety, sleep, hormonal secretion, feeding behavior, locomotor activity, as well as learning and memory processes. 5-HT2CR dysfunction has been implicated in pathological conditions, including anxiety and depression.

Background art includes Papp et al., Front. Pharmacol. 10:1636. doi: 10.3389/fphar.2019.01636 and US Patent Application No. 20020177596.

### SUMMARY OF THE INVENTION

According to an aspect of the present invention there is provided a method of treating a disease or disorder for which an increase in immune cells in the brain is therapeutic in a subject in need thereof, comprising administering to the subject a therapeutically effective amount of an agent which specifically binds and increases activity of a receptor selected from the group consisting of a 5HT2C receptor, a 5HT2A receptor and a 5HT2B receptor in the postcentral gyrus and/or the choroid plexus (CP) of the brain of the subject, thereby treating the disease, with the proviso that the disease is not depression or anxiety.

According to another aspect of the present invention there is provided an agent which specifically binds and increases activity of a receptor selected from the group consisting of a 5HT2C receptor, a 5HT2A receptor and a 5HT2B receptor in the postcentral gyrus and/or the choroid plexus (CP) of the brain of a subject for use in treating a disease or disorder for which an increase in immune cells in the brain is therapeutic for a subject, with the proviso that the disease is not depression or anxiety.

According to another aspect of the present invention there is provided a method of treating a disease or disorder for which an increase in immune cells in the brain is therapeutic in a subject in need thereof, comprising administering to the subject a therapeutically effective amount of an agent which increases the amount of serotonin in the brain of the subject, thereby treating the disease, with the proviso that the disease is not depression or anxiety.

According to another aspect of the present invention there is provided an agent which increases the amount of serotonin in the brain of the subject for use in treating a disease or disorder for which an increase in immune cells in the brain is therapeutic for a subject, with the proviso that the disease is not depression or anxiety.

According to another aspect of the present invention there is provided a method of treating a disease or disorder for which a decrease in immune cells in the brain is therapeutic in a subject in need thereof, comprising administering to the subject a therapeutically effective amount of an agent which specifically binds and reduces activity of a receptor selected from the group consisting of a 5HT2C receptor, a 5HT2A receptor and a 5HT2B receptor in the postcentral gyrus and/or the choroid plexus (CP) of the brain of the subject, thereby treating the disease.

According to another aspect of the present invention there is provided an agent which specifically binds and reduces activity of a receptor selected from the group consisting of a 5HT2C receptor, a 5HT2A receptor and a 5HT2B receptor in the postcentral gyrus and/or the choroid plexus (CP) of the brain of a subject for use in treating a disease or disorder for which a decrease in immune cells in the brain is therapeutic.

According to another aspect of the present invention there is provided an article of manufacture comprising a first agent which specifically binds and increases activity of a 5HT2C receptor; and at least one additional agent selected from the group consisting of a selective serotonin reuptake inhibitor (SSRI), a serotonin and norepinephrine reuptake inhibitor (SNRI), bupropion, a tricyclic antidepressant, a monoamine oxidase inhibitor (MAOI) and an agent which specifically targets and activates a receptor selected from the group consisting of 5HT1B, 5HT2A and 5HT2B.

According to some embodiments of the invention, the receptor is a 5HT2C receptor.

According to some embodiments of the invention, the disease is a neurodegenerative disease.

According to some embodiments of the invention, the disease is a mood disorder.

According to some embodiments of the invention, the disease is selected from the group consisting of schizophrenia, post-traumatic stress disorder (PTSD), epilepsy, autism, stroke, ischemia, Alzheimer's disease, Parkinson's disease, migraine and cancer.

According to some embodiments of the invention, the cancer is a brain cancer.

According to some embodiments of the invention, the agent is selected from the group consisting of Lorcaserin, Vabisacerin, RO 60-01752, YM-348, mCPP, CP809101, WAY163909 and ORG12962.

According to some embodiments of the invention, the agent is an activating antibody.

According to some embodiments of the invention, the method further comprises administering to the subject an additional agent which specifically targets and activates a receptor selected from the group consisting of 5HT1B, 5HT2A and 5HT2B.

According to some embodiments of the invention, the method further comprises administering to the subject an additional agent selected from the group consisting of a selective serotonin reuptake inhibitor (SSRI), a serotonin and norepinephrine reuptake inhibitor (SNRI), bupropion, a tricyclic antidepressant and a monoamine oxidase inhibitor (MAOI).

According to some embodiments of the invention, the agent is selected from the group consisting of a selective serotonin reuptake inhibitor (SSRI), a serotonin and norepinephrine reuptake inhibitor (SNRI), bupropion, a tricyclic antidepressant and a monoamine oxidase inhibitor (MAOI).

According to some embodiments of the invention, the disease is an autoimmune disease.

According to some embodiments of the invention, the autoimmune disease is Multiple Sclerosis.

According to some embodiments of the invention, the agent is an antibody.

According to some embodiments of the invention, the receptor is a 5HT2C receptor.

According to some embodiments of the invention, the antibody is selected from the group consisting of NBP3-12279, NBP2-67100, NB100-1524, MBS555177 and BS-2959R.

According to some embodiments of the invention, the agent is a 5HT2C antagonist.

According to some embodiments of the invention, the antagonist is selected from the group consisting of RS-102221, SB-221284, SB242084, FR260010 and SDX-SER-082.

According to some embodiments of the invention, the method further comprises administering to the subject a therapeutically effective amount of an additional agent selected from the group consisting of a selective serotonin reuptake inhibitor (SSRI), a serotonin and norepinephrine reuptake inhibitor (SNRI), bupropion, a tricyclic antidepressant and a monoamine oxidase inhibitor (MAOI).

According to some embodiments of the invention, the first agent and the at least one additional agent are formulated in a single pharmaceutical composition.

According to some embodiments of the invention, the first agent and the at least one additional agent are formulated in separate compositions.

According to some embodiments of the invention, the first agent is selected from the group consisting of Lorcaserin, Vabisacerin, RO 60-01752, YM-348, mCPP, CP809101, WAY163909 and ORG12962.

According to some embodiments of the invention, the first agent is an activating antibody.

According to some embodiments of the invention, the article of manufacture is for use in treating a disease or disorder for which an increase in immune cells in the brain is therapeutic.

According to some embodiments of the invention, the disease is not depression or anxiety.

According to some embodiments of the invention, the disease is a neurodegenerative disease.

According to some embodiments of the invention, the disease is a mood disorder. According to some embodiments of the invention, the disease is selected from the group consisting of schizophrenia, post-traumatic stress disorder (PTSD), epilepsy, autism, stroke, ischemia, Alzheimer's disease, Parkinson's disease, migraine and cancer.

According to some embodiments of the invention, the cancer is a brain cancer.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-L. Serotonergic activation increases migration of CD45^{high} cells to the brain compartment. A. Representative image of activated neurons expressing the neuronal activation marker cFos (green) in the DR of control vs. Gq (DAPI-white; scale bar 100 mm). B. Representative image of activated serotonergic neurons (mCherry-red) in the DR of DREADD-injected Gq mice. The activated serotonergic neurons co-express the cFos activation marker (green) and the mCherry fluorescence DREADD reporter (red) (DAPI-white; scale bar 50 mm). C. Fold increase of cFos expressing cells in the DR area (mm²) in control and Gq mice. D. Representative histogram plot of FACS analysis demonstrating the CD45⁺ cells in the brain compartment, 90 minutes following activation with CNO, in control (gray) vs. Gq (blue) mice. E. Fold increase of CD45^{high} cells out of total CD45+ cells in the brains of control (gray) vs. Gq (blue) mice. F. SPADE plot of CyTOF analysis demonstrating the expression of CD45 in the brain compartment of control (left) and Gq (right) mice. Intensity of CD45 expression is indicated by the colored bar. Size of nodes represent the number of cells assigned to each node. G and H. tSNE plots of CyTOF analysis of CD11b and MHCII expression, respectively, in the brains of control and Gq mice. I. Representative pseudocolor plot of CD11b⁺CD45⁺ in control vs. Gq brain cells. J. Fold increase of brain CD11b⁺CD45^{high} cells in control vs. Gq mice. K. Representative pseudocolor plot of MHCII⁺CD45⁺ cells in control vs. Gq brain cells. L. Fold increase of brain MHCII+ cells from CD45^{high} cells in control vs. Gq mice. Data are represented as mean±SEM.

FIGS. 2A-F. Immune cells enter the brain through the 3^{rd} ventricle. A. Representative image of CD45⁺ cells (green) in the LV from control and Gq mice (scale bar 50 mm). B. Fold change of CD45⁺ cells in LV of control vs. Gq mice. C. Representative image of CD45⁺ cells (green) in the 3V from control and Gq mice (scale bar 50 mm). D. Fold increase of CD45⁺ cells in 3V of control vs. Gq mice. E. Representative image of CD45⁺ cells (green) in the 4V from control and Gq mice (scale bar 50 mm). F. Fold change of CD45⁺ cells in 4V of control vs. Gq mice. LV—lateral ventricle, 3V—third ventricle, 4V—fourth ventricle. Data are represented as mean±SEM.

FIGS. 3A-C. Morphological and immunological characterization of the PG. A. Representative micrographs of extracted meninges with PG, stained with CD45 for immune cells (green), GFAP for astrocytes (purple), and a merged image (scale bar 1000 mm. In the enlarged images bar is 50 mm). B. Representative z-stack images of 30 (bar 100 mm), 60 (scale bar 100 mm) and 90 (scale bar 50 mm) minutes after serotonergic activation in control vs. Gq mice. The 90 minutes images following serotonergic activation are sagittal sections from the brain attached to the skull, while the images from 30 and 60 minutes are taken from the gland itself using z-stack. C. Quantification of CD45^{high} cells as an average of three adjacent areas from total area (mm²) of the sPG at 0, 30 (n=3), 60 (n=3) and 90 (n=5) minutes after serotonergic activation (CNO administration) in control vs. Gq mice. Dashed line indicates normalization to controls. Data are represented as mean±SEM. olf-olfactory, LV-lateral ventricle, 3V-3^{rd} ventricle, PG-pineal gland, Cer-cerebellum, 4V-4^{th} ventricle, sPG-superficial pineal gland, dPG-deep pineal gland, Pr-pineal recess.

FIGS. 4A-D. MERFISH spatial transcriptomics and single-cell characterization of the PG. A. UMAP representation of all clustered cells in a mid-sagittal section of the brain from MERFISH data analysis. B. Spatial plot with the cell type positions in the brain including C. zoomed-in panel of the pineal gland area D. Dot plot showing the average expression levels of the major cell type markers per cell cluster (OPCs—oligodendrocyte progenitor cells).

FIGS. 5A-D. 5-HT_{2C} expression in PG area. A. Violin plots from PG single cells showing the expression (log-normalised, 0 values excluded) of all 18 serotonin receptors within the scRNA-seq data. B. Violin plots showing the highest expression level of Htr2c in cluster 10 then 2, across all clusters in scRNA-seq data. C. MERFISH spatial plots showing expression of two genes encoding serotonin receptors Htr2a and Htr2c in the pineal gland area. D. Representative IHC staining of 5-HT_{2C} (red) and astrocytes (GFAP-blue) in the sPG and dPG areas, and merge (bar 50 mm).

FIGS. 6A-F. Serotonergic pharmacological manipulation affects immune cell infiltration to the brain. A. Representative pseudocolor plot of brain CD45^{high} cells in control, 5-HT_{2B} receptor and 5-HT_{2C} receptor agonists-treated mice. B. Fold increase of CD45^{high} cells measured by FACS, in the brain compartment of control and 5-HT_{2B} receptor agonist and 5-HT_{2C} receptor agonist treated mice. The brains were analyzed 2 hours after administration of the serotonergic agonists. C. Schematic representation of SI model and treatment duration. D. Representative pseudocolor plot of CD45^{high} cells in the brains of control, saline, Fluoxetine (FLX), FLX+5-HT2c antagonist treated mice that underwent the social isolation (SI) model (described previously in FIG. 6C). E. Fold increase of CD45^{high} cells in brain compartment measured by FACS in SI—saline, SI—FLX and SI—FLX+5-HT_{2c} antagonist treated mice compared to control mice. F. Fold change of immobility in tail suspension test (TST) between SI—saline, SI—FLX and SI—FLX+5-HT_{2c} treated mice compared to control mice. Data are represented as mean±SEM.

### DESCRIPTION OF SPECIFIC EMBODIMENTS OF THE INVENTION

The present invention, in some embodiments thereof, relates to methods of treating neurodegenerative, mental, cognitive and autoimmune disorders using agent which specifically target the 5-HT_{2C} receptor.
Before explaining at least one embodiment of the invention in detail, it is to be understood that the invention is not necessarily limited in its application to the details set forth in the following description or exemplified by the Examples.

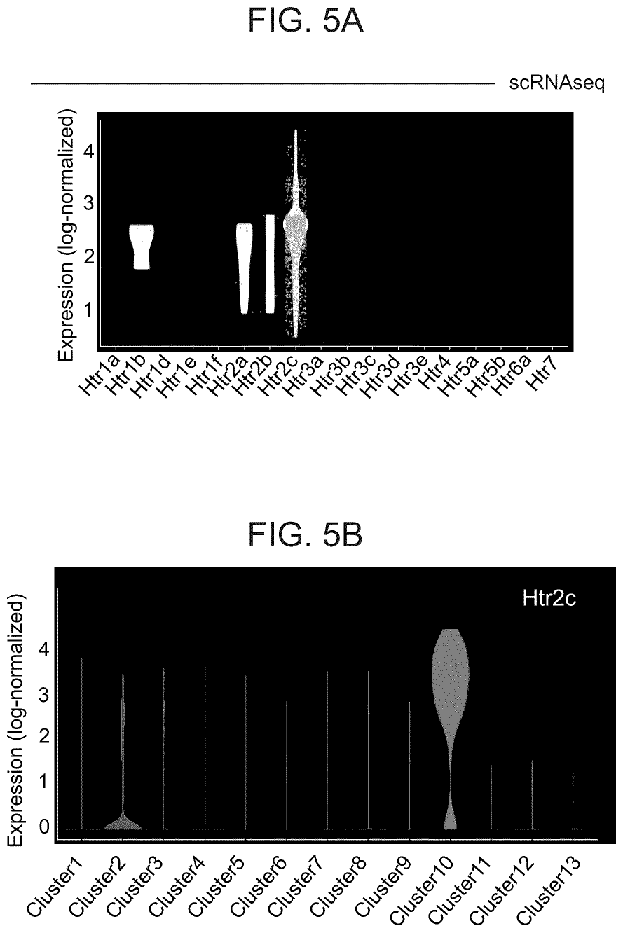

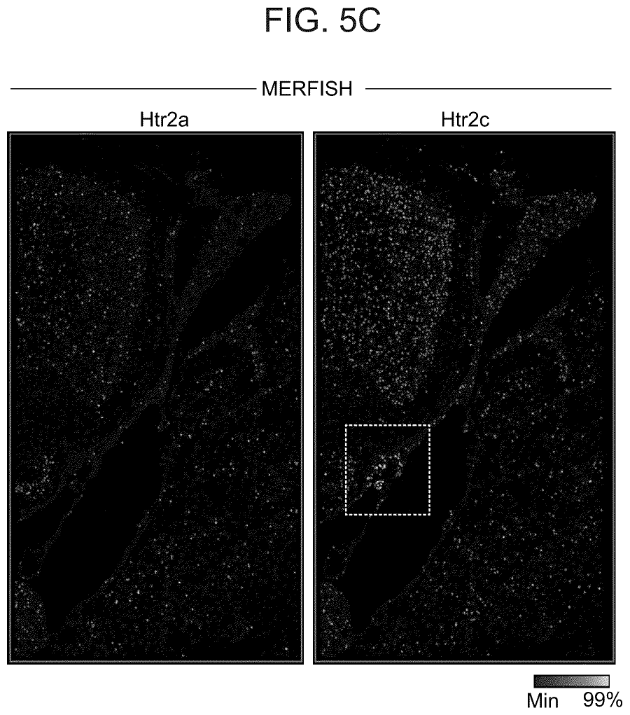

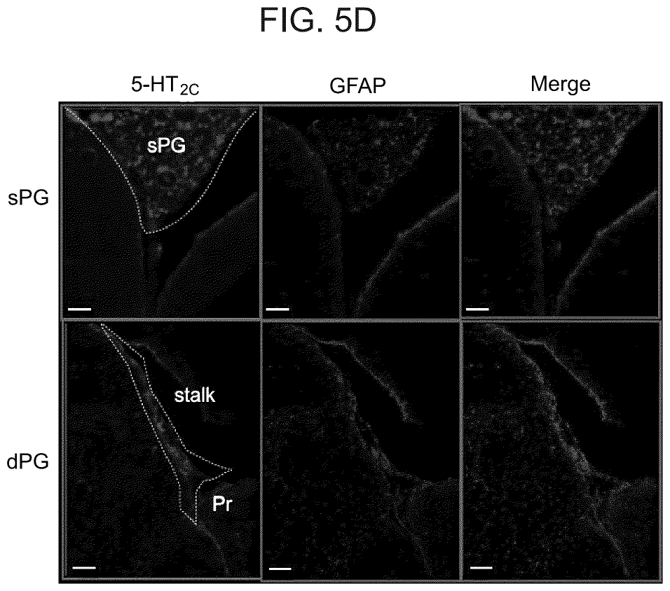

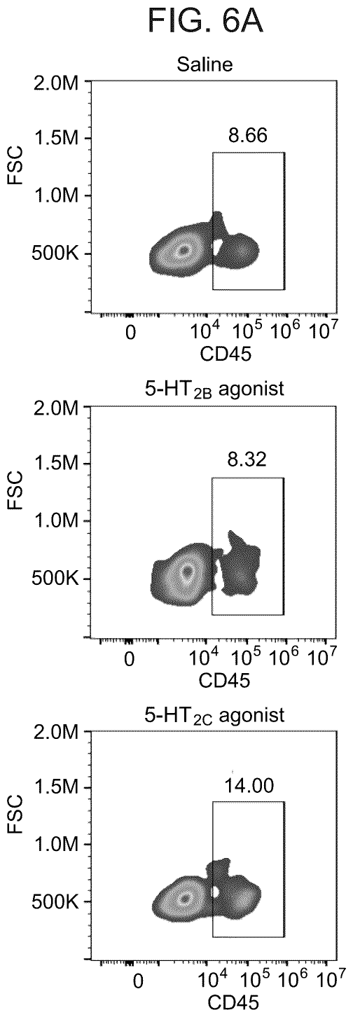

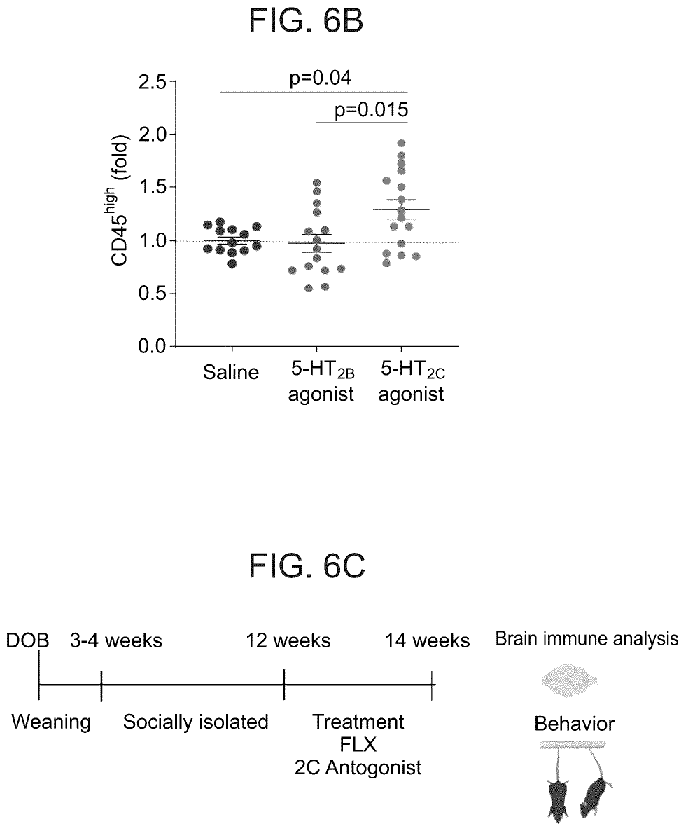

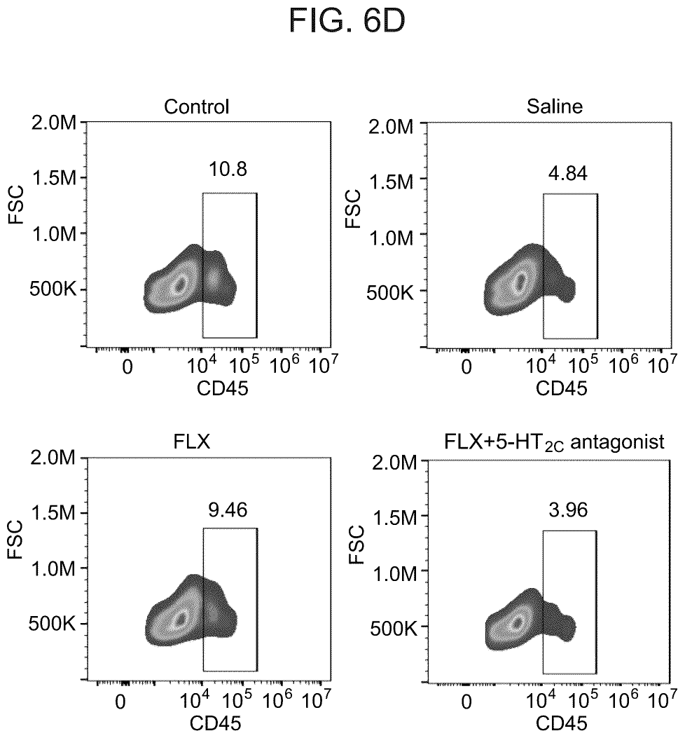

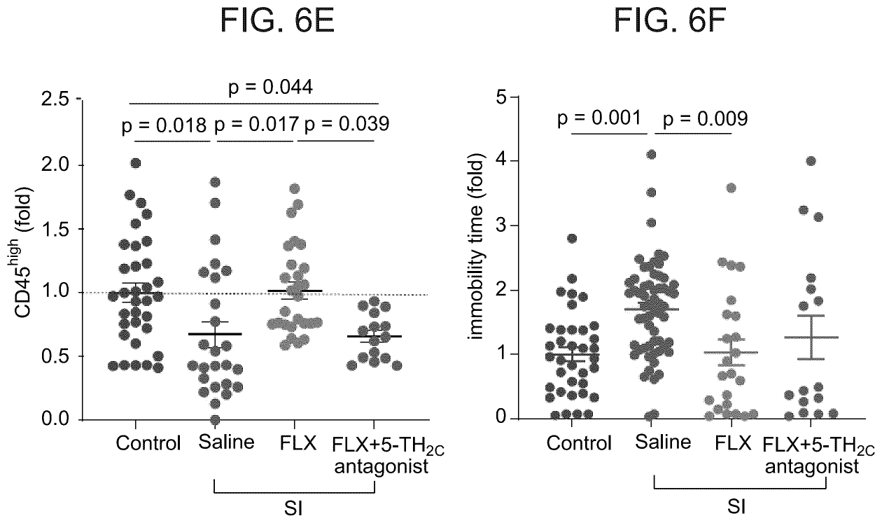

The invention is capable of other embodiments or of being practiced or carried out in various ways. Peripheral immune cells play a critical role in brain activity, but the mechanisms controlling the infiltration of these cells to the brain, remain largely unknown. The present inventors have now shown in mice that serotonin can regulate the immune infiltration processes. Chemogenetic activation of dorsal raphe serotonergic neurons was sufficient to induce immune cell infiltration, primarily into the 3rd ventricle. Through spatial transcriptomics and single-cell genomics, it was found that the pineal gland, which is connected the 3rd ventricle, is enriched with the serotonergic receptor, 5-HT2C (FIGS. 5A-D). Notably, pharmacological activation of this receptor and treatment with selective serotonin reuptake inhibitor (SSRI) induced a peripheral immune infiltration (FIGS. 6A-B). Blocking the 5-HT2C receptor abolished these effects and attenuated the functional benefits of the SSRI (FIGS. 6D-E). Collectively, these findings introduce serotonin as the brain's “gate-keeper” in regulating its immune composition relevant for treating autoimmune disorders, neurodegeneration and depression.

Thus, according to a first aspect of the present invention, there is provided a method of treating a disease or disorder for which an increase in immune cells in the brain is therapeutic in a subject in need thereof, comprising administering to the subject a therapeutically effective amount of an agent which specifically binds and increases activity of a receptor selected from the group consisting of a 5HT2C receptor, a 5HT2A receptor and a 5HT2B receptor in the postcentral gyrus and/or the choroid plexus (CP) of the brain of the subject, thereby treating the disease, with the proviso that the disease is not depression or anxiety.

According to one embodiment, the agent binds specifically to 5-HT_{2C} receptor which is expressed in the postcentral gyrus and/or the choroid plexus (CP) of the brain. The phrase “specifically bind(s)” or “bind(s) specifically” when referring to a binding molecule refers to a binding molecule which has intermediate or high binding affinity, exclusively or predominately, to a target molecule, such as to 5-HT_{2C} receptor. The phrase “specifically binds to” refers to a binding reaction which is determinative of the presence of a target protein (such as 5-HT_{2C} receptor) in the presence of a heterogeneous population of proteins and other biologics. Thus, under designated assay conditions, the specified binding molecules bind preferentially to a particular target protein (e.g. 5-HT_{2C} receptor) and do not bind in a significant amount to other components present in a test sample. Specific binding to a target protein under such conditions may require a binding molecule that is selected for its specificity for a particular target protein. A variety of assay formats may be used to select binding molecules that are specifically reactive with a particular target protein. For example, solid-phase ELISA immunoassays, immunoprecipitation, Biacore and Western blot may be used to identify binding molecules that specifically bind to 5-HT_{2C} receptor. Typically, a specific or selective reaction will be at least twice background signal or noise and more typically more than 10 times background. Typically, an agent that specifically binds the 5-HT_{2C} receptor binds the receptor with a dissociation constant (K_{D}) of at least about 1×10⁻⁶ to 1×10⁻⁷, or about 1×10⁻⁸ to 1×10⁻⁹ M, or about 1×10⁻¹⁰ to 1×10⁻¹¹ or higher; and/or binds to the 5-HT_{2C} receptor with an affinity that is at least two-fold, five-fold, ten-fold, twenty-fold greater than its affinity for binding to a non-specific antigen (e.g., BSA, casein) or to at least one, two or all of the following receptors: 5HT1B, 5HT2A and 5HT2B.

In one embodiment, the agent is an agonist which specifically targets the 5-HT_{2c} receptor.

Exemplary 5-HT_{2C} agonists include Lorcaserin, vabisacerin, RO 60-01752, YM-348, mCPP, CP809,101, WAY163909 and ORG12962. Additional information regarding these agonists is found in Table 1, herein below.

TABLE 1

| | S-HT_{2C} | | 5-HT_{2A} | | 5-HT_{2B} | | |
|---|---|---|---|---|---|---|---|
| | EC₅₀ | Eₘₐₓ | EC₅₀ | Eₘₐₓ | EC₅₀ | Eₘₐₓ | |
| Compound | [n_{M}] | [%] | [n_{M}] | [%] | [n_{M}] | [%] | Ref. |
| RO 60- | 52 ± 3 | 88 ± 20 | 400 ± 20 | 91 ± 5 | 2.4 ± 1 | 130 ± 30 | [¹⁰] |
| 01752 | | | | | | | |
| YM-348 | 1.0 ± 0.2 | 76 ± 1 | 93 ± 10 | 97 ± 2 | 3.2 ± 3 | 110 ± 10 | [¹⁰] |
| mCPP | 120 ± 10 | 63 ± 3 | 150 ± 20 | 18 ± 2 | 93 ± 50 | 21 ± 9 | [¹⁰] |
| CP-809101 | 0.11 | 93 | 153 | 67 | 65.3 | 57 | [¹³] |
| Lorcaserin | 9 ± 0.5 | 100 | 168 ± 11 | 75 | 943 ± 90 | 100 | [¹⁵] |
| Vabicaserin | 8 | 100 | 1650 | —^{[a]} | 1.5^{[b]} | 80 | [¹⁶] |
| | | | | | >10000^{[c]} | | |
| WAY- | 8 ± 3 | 90 ± 6 | NE | NE | 185 ± 105 | 40 ± 3 | [¹⁷] |
| 163909 | | | | | | | |

As well as administering an agent that activates the 5HT2C receptor, the present inventors further contemplate administering additional agents to the subject that specifically target and activate a 5HT1B, 5HT2A and/or 5HT2B receptors.

Such 5HT agonists are provided in Tables 2 and 3. It will be appreciated that depending on the dose of the agents, in some instances the agents may act as selective agonists/antagonists at particular 5HT receptors.

TABLE 2

| Compound | Pharmacology of 5-HT_{2C} Receptors | |
|---|---|---|
| Name | Chemical Name | Actions |
| Agomelatine | N-[2-(7-methoxynaphthalen-1-yl)ethyl] | 5-HT [symbol] |
| (S-20098) | acetamide | antagonist/melatonin |
| | | agonist |
| BVT-933 | Undisclosed | 5-HT [symbol] agonist |
| (PRX-00933) | | |
| CP-809101 | 2-(3-Chlorobenzyloxy)-6-(piperazin-1- | 5-HT [symbol] agonist |
| | yl)pyrazine | |
| Lorcaserin | (1R)-8-chloro-2,3,4,5-tetrahydro-1-methyl- | 5-HT [symbol] agonist |
| (APD-356) | 1H-3-benzazepine | |
| mCPP | m-Chlorophenylpiperazine | Dual 5-HT [symbol] agonist |
| RO60-0175 | (S)-2-(6-chloro-5-fluoroindol-1-yl)-1- | 5-HT [symbol] agonist |
| | methylethylamine | |
| RO60-0491 | (S)-2-(4,4,7-trimethyl-1,4-dihydro- | 5-HT [symbol] antagonist |
| | indeno[1,2-b]pyrrol-1-yl)-1- | |
| | methylethylamine | |
| S-32006 | N-pyridin-3-yl-1,2-dihydro-3H- | 5-HT [symbol] antagonist |
| | benzo[e]indole-3-carboxamide | |
| SB-206533 | 5-Methyl-1-(3- [symbol] )-1,2,3,5- | 5-HT [symbol] antagonist/5- |
| | tetrahydropyrrolo[2,3- [symbol] indole | HT [symbol] inverse agonist |
| SB-242084 | 6-Chloro-5-methyl-1-[[2-[(2-methyl-3- | 5-HT [symbol] antagonist |
| | pyridyl)oxy]-5-pyridyl]carbamoyl]indoline | |
| SB-243213 | 5-Methyl-1-[[2-[(2-methyl-3-pyridyl)oxy]- | 5-HT [symbol] inverse agonist |
| | 5-pyridyl]carbamoyl]-6- | |
| | trifluoromethylindoline | |
| Vabicaserin | (9aR,12aS)-4,5,6,7,9,9a,10,11,12,12a- | 5-HT [symbol] agonist |
| (SCA-136) | decahydrocyclopenta[c][1,4]diazepino[6,7, | |
| | [symbol] quinoline | |
| WAY-161503 | (4aR)-8,9-dichloro-2,3,4,4a-tetrahydro-1H- | Dual 5-HT [symbol] agonist |
| | pyrazino[1,2-a]quinoxalin-5(6H)-one | |
| WAY-163909 | (7bR,10aR)-1,2,3,4,8,9,10,10a-octahydro- | 5-HT [symbol] agonist |
| | 7bH-cyclopenta-[b][1,4]diazepino[6,7,1hi] | |
| | indole | |

[symbol]

indicates data missing or illegible when filed

TABLE 3

| Name | Agonist | Antagonist | Syndrome |
|---|---|---|---|
| ADDYI | 1A | 2A > 2B, 2C, D4 | Hypoactive Sexual Desire Disorder |
| Amerge | 5-HT1B/1D | migraine | |
| Axert | 1B/1D | | migraine |
| Frova | 1B/1D | | migraine |
| Imitrex, | 1B/1D | | migraine |
| TOSYMRA, | | | |
| TREXIMET | | | |
| Maxalt | 1B/1D | | migraine |
| Naratriptan | 1B/1D | | migraine |
| Relpax | 1B/1D | | migraine |
| ZOMIG | B/1D | | migraine |
| BELVIQ | 2c | | chronic weight management |
| ZOFRAN | | 5ht3 | nausea and vomiting |
| SUSTOL | | 5ht3 | nausea and vomiting |
| Alosetron | | 5ht3a | severe diarrhoea-predominant irritable |
| | | | bowel syndrome (IBS) in women |
| trazadone | | 5HT2a | depression, insomnia, anxiety |
| Nefazodone | | 5HT2a | depression, insomnia, anxiety |

According to a particular embodiment, the agent which specifically binds and increases activity of the 5HT2C receptor, the 5HT2A receptor and/or the 5HT2B receptor in the postcentral gyrus and/or the choroid plexus (CP) is an activating antibody.

The term “antibody” as used in this invention includes intact molecules as well as functional fragments thereof, such as Fab, F(ab′)2, Fv or single domain molecules such as VH and VL to an epitope of an antigen. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; (5) Single chain antibody (“SCA”), a genetically engineered molecule containing the variable region of the light chain and the variable region of the heavy chain, linked by a suitable polypeptide linker as a genetically fused single chain molecule; and (6) Single domain antibodies are composed of a single VH or VL domains which exhibit sufficient affinity to the antigen.

In a particular embodiment, the antibody is a monoclonal antibody.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference and the Examples section which follows).

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

In addition to administering to the subject an agent which binds to and activates one of the receptors 5HT2C, HT2A or 5HT2B, the present inventors further contemplate administering to the subject an additional agent that increases serotonin in the brain (which does not bind specifically to one of the above mentioned receptors). Such agents include, but are not limited to selective serotonin reuptake inhibitors (SSRI), serotonin and norepinephrine reuptake inhibitors (SNRI), bupropion, tricyclic antidepressants and monoamine oxidase inhibitors (MAOI).

Examples of SSRIs include, but are not limited to Citalopram (Celexa), Escitalopram (Lexapro), Fluoxetine (Prozac), Fluvoxamine (Luvox), Paroxetine (Paxil) and Sertraline (Zoloft).

Examples of SNRIs include, but are not limited to Desvenlafaxine (Pristiq), Duloxetine (Cymbalta), Levomilnacipran (Fetzima) and Venlafaxine (Effexor XR).

Examples of tricyclic antidepressants that inhibit the reuptake of serotonin include, but are not limited to Butriptyline (Evadyne), Clomipramine (Anafranil), Imipramine (Tofranil, Janimine, Praminil) and Trimipramine (Surmontil).

Examples of monoamine oxidase inhibitors include but are not limited to Hydrazine, Isocarboxazid (Marplan), Hydracarbazine, Phenelzine (Nardil, Nardelzine), Tranylcypromine (Parnate, Jatrosom), Bifemelane (Alnert, Celeport) Methylthioninium chloride (Urelene blue, Provayblue, Proveblue), Moclobemide (Aurorix, Manerix, Moclamine), Pirlindole (Pirazidol), Rasagiline (Azilect) and Selegiline (Deprenyl, Eldepryl, Emsam, Zelapar).

Such additional agents may be formulated together with the agents that bind to and activate one of the receptors 5HT2C, HT2A or 5HT2B or may be provided as separate formulations as further described herein below.

In one embodiments, activation of any one of the receptors 5HT2C, HT2A or 5HT2B leads to an increase in serotonin in the postcentral gyrus and/or the choroid plexus (CP).

Thus, according to another aspect of the present invention there is provided a method of treating a disease or disorder for which an increase in immune cells in the brain is therapeutic in a subject in need thereof, comprising administering to the subject a therapeutically effective amount of an agent which increases the amount of serotonin in the brain of the subject, thereby treating the disease.

Examples of agents which increase serotonin in the brain include selective serotonin reuptake inhibitors (SSRI), serotonin and norepinephrine reuptake inhibitors (SNRI), bupropion, tricyclic antidepressants and monoamine oxidase inhibitors (MAOI), as further described herein above. Additional agents include those that bind to and activate any one of the receptors 5HT2C, HT2A or 5HT2B in the postcentral gyrus and/or the choroid plexus (CP).

Diseases for which an increase in immune cells in the brain is therapeutic include neurodegenerative diseases.

The term “neurodegenerative disease” is used herein to describe a disease which is caused by damage to the central nervous system. Exemplary neurodegenerative diseases which may be treated using the cells and methods according to the present invention include for example: Amyotrophic Lateral Sclerosis (ALS), Parkinson's disease, Multiple System Atrophy (MSA), Huntington's disease, Alzheimer's disease, Rett Syndrome, lysosomal storage diseases (“white matter disease” or glial/demyelination disease, as described, for example by Folkerth, J. Neuropath. Exp. Neuro., September 1999, 58:9), including Sanfilippo, Gaucher disease, Tay Sachs disease (beta hexosaminidase deficiency), other genetic diseases, multiple sclerosis (MS), brain injury or trauma caused by ischemia, accidents, environmental insult, etc., spinal cord damage, ataxia. In addition, the present invention may be used to reduce and/or eliminate the effects on the central nervous system of a stroke in a patient, which is otherwise caused by lack of blood flow or ischemia to a site in the brain of the patient or which has occurred from physical injury to the brain and/or spinal cord. Neurodegenerative diseases also include neurodevelopmental disorders including for example, autism and related neurological diseases such as schizophrenia, among numerous others.

Additional diseases for which an increase in immune cells in the brain is therapeutic include mood disorders (with the proviso that the mood disorder is not depression or anxiety).

Still other diseases for which an increase in immune cells in the brain is therapeutic include schizophrenia, post-traumatic stress disorder (PTSD), epilepsy, autism, stroke, ischemia, Alzheimer's disease, Parkinson's disease, migraine and cancer (e.g. brain cancer).

According to embodiments of the invention, the cancer is a brain-metastasized cancer including but not limited to brain-metastasized melanoma, brain-metastasized breast cancer, brain-metastasized lung cancer, or brain-metastasized colorectal cancer.

In one embodiment, the brain cancer is glioblastoma.

As used herein, the term “glioblastoma” (GBM), also called glioblastoma multiforme or “grade IV astrocytoma” according to WHO classification refers to a central nervous system primary tumor derived from glial cells. GBM is one of the deadliest human cancers with an incidence of about 3.5/100,000 per year worldwide (Cloughesy, T. F., W. K. Cavenee, and P. S. Mischel, Glioblastoma: from molecular pathology to targeted treatment. Annu Rev Pathol, 2014. 9: p. 1-25). Despite the aggressive standard of care currently used including surgery, chemo- and radiotherapy, the prognosis remains very poor with about 15 months overall survival.

According to a particular embodiment, the glioblastoma is at an early stage (e.g. when the tumor is of a diameter of less than 14 mm).

According to still another aspect of the present invention, there is provided a method of treating a disease or disorder for which a decrease in immune cells in the brain is therapeutic in a subject in need thereof, comprising administering to the subject a therapeutically effective amount of an agent which specifically binds and reduces activity of a receptor selected from the group consisting of a 5HT2C receptor, a 5HT2A receptor and a 5HT2B receptor in the postcentral gyrus and/or the choroid plexus (CP) of the brain of the subject, thereby treating the disease.

Exemplary agents that bind to and reduce activity of any one of the above mentioned receptors include antagonists which selectively bind at least one of these receptors (e.g. 5HT2C receptor).

Exemplary antagonists of 5HT2C receptor, 5HT2A receptor or 5HT2B receptors include RS-102221, SB-221284, SB242084, FR260010 and SDX-SER-082. Additional antagonists are listed in Tables 2 and 3 herein above.

Other exemplary agents that bind to and reduce activity of any one of the above mentioned receptors include inhibitory antibodies which specifically target one of these receptors. Examples of such antibodies include of NBP3-12279, NBP2-67100, NB100-1524, MBS555177 and BS-2959R.

Diseases for which a decrease in immune cells in the brain is therapeutic include autoimmune diseases of the nervous system. Such diseases include for example, multiple sclerosis and myasthenia gravis, Guillain bar syndrome, Multiple system Atrophy (MSA; a sporadic, progressive, adult-onset neurodegenerative disorder associated with varying degrees of parkinsonism, autonomic dysfunction and cerebellar ataxia). Other autoimmune diseases are described in Kraker et al., Curr Neuropharmacol. 2011 September; 9(3): 400-408, the contents of which are incorporated herein by reference.

It will be appreciated that the agent of the present invention (e.g., the agonist or the antibody) can be administered to the subject per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the agent of the present invention which is accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include oral, rectal, neurosurgical strategies (e.g., intracerebral injection, intrastriatal infusion or intracerebroventricular infusion, intra spinal cord, epidural), transmucosal, intestinal or parenteral delivery, including intramuscular, subcutaneous and intramedullary injections as well as intrathecal, direct intraventricular, intracardiac, intravenous, intraperitoneal, intranasal, or intraocular injections.

Alternately, one may administer the pharmaceutical composition in a local rather than a systemic manner, for example, via injection of the pharmaceutical composition directly into a tissue region of a patient (e.g. into the brain).

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide).

Additional strategies to cross the blood brain barrier are reviewed by Bellettato and Scarpa Italian Journal of Pediatrics 2018, 44(Suppl 2):131, the contents of which are incorporated herein by reference.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran.

Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose (e.g. treat the disease for which an increase/decrease in immune cells in the brain is therapeutic).

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide tissue levels of the active ingredient that are sufficient to decrease the number or size of adipocytes or decrease visceral fat (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions for human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

As mentioned, the present inventors contemplate administration of an agent which specifically binds and increases activity of a 5HT2C receptor together with at least one additional agent selected from the group consisting of a selective serotonin reuptake inhibitor (SSRI), a serotonin and norepinephrine reuptake inhibitor (SNRI), bupropion, a tricyclic antidepressant, a monoamine oxidase inhibitor (MAOI) and an agent which specifically targets and activates a receptor selected from the group consisting of 5HT1B, 5HT2A and 5HT2B.

Thus, according to still another aspect of the present invention there is provided an article of manufacture comprising a first agent which specifically binds and increases activity of a 5HT2C receptor; and at least one additional agent selected from the group consisting of a selective serotonin reuptake inhibitor (SSRI), a serotonin and norepinephrine reuptake inhibitor (SNRI), bupropion, a tricyclic antidepressant, a monoamine oxidase inhibitor (MAOI) and an agent which specifically targets and activates a receptor selected from the group consisting of 5HT1B, 5HT2A and 5HT2B.

The first agent and the additional agent may be packaged separately in the article of manufacture or packaged in a single package. In one embodiment, the first agent and the additional agent are formulated in a single formulation.

Such combinations are contemplated for a myriad of uses including for example for treating a disease or disorder for which an increase in immune cells in the brain is therapeutic. In one embodiment, the combinations are not for treating depression or anxiety.

Examples of particular diseases which can be treated using the article of manufacture disclosed herein include schizophrenia, post-traumatic stress disorder (PTSD), epilepsy, autism, stroke, ischemia, Alzheimer's disease, Parkinson's disease, migraine and cancer (e.g. brain cancer), as further detailed herein above.

In the context of a combination therapy, the first agent may be administered by the same route of administration (e.g. centrally) as the additional agent is administered. In the alternative, the first agent may be administered by a different route of administration to the additional agent.

The chemotherapeutic agent can be administered immediately prior to (or after) the Additional agent, on the same day as, one day before (or after), one week before (or after), one month before (or after), or two months before (or after) the additional agent, and the like.

The first agent and the additional agent can be administered concomitantly, that is, where the administering for each of these reagents can occur at time intervals that partially or fully overlap each other. The first agent and the additional agent can be administered during time intervals that do not overlap each other. For example, the first agent can be administered within the time frame of t=0 to 1 hours, while the additional agent can be administered within the time frame of t=1 to 2 hours. Also, the first agent can be administered within the time frame of t=0 to 1 hours, while the additional agent can be administered somewhere within the time frame of t=2-3 hours, t=3-4 hours, t=4-5 hours, t=5-6 hours, t=6-7 hours, t=7-8 hours, t=8-9 hours, t=9-10 hours, and the like. Moreover, the additional agent can be administered somewhere in the time frame of t=minus 2-3 hours, t=minus 3-4 hours, t=minus 4-5 hours, t=5-6 minus hours, t=minus 6-7 hours, t=minus 7-8 hours, t=minus 8-9 hours, t=minus 9-10 hours.

The additional agent of the present invention and the first agent are typically provided in combined amounts to achieve therapeutic and/or prophylactic effectiveness. This amount will evidently depend upon the particular compound selected for use, the nature and number of the other treatment modality, the condition(s) to be treated, prevented and/or palliated, the species, age, sex, weight, health and prognosis of the subject, the mode of administration, effectiveness of targeting, residence time, mode of clearance, type and severity of side effects of the pharmaceutical composition and upon many other factors which will be evident to those of skill in the art. The additional agent will be used at a level at which therapeutic, and/or prophylactic effectiveness in combination with the first agent is observed.

The first agent may be administered (together with the additional agent) at a gold standard dosing as a single agent, below a gold standard dosing as a single agent or above a gold standard dosing as a single agent.

According to specific embodiments, the first agent is administered below the gold standard dosing as a single agent.

As used herein the term “gold standard dosing” refers to the dosing which is recommended by a regulatory agency (e.g., FDA), for a given disease at a given stage.

According to other specific embodiments, the first agent is administered at a dose that does not exert at least one side effect which is associated with the gold standard dosing.

Thus, in one embodiment, the amount of the first agent is below the minimum dose required for therapeutic and/or prophylactic effectiveness when used as a single therapy (e.g. 10-99%, preferably 25 to 75% of that minimum dose). This allows for reduction of the side effects caused by the first agent but the therapy is rendered effective because in combination with the additional agent, the combinations are effective overall.

In one aspect of the present invention, the additional agent and the first agent are synergistic with respect to their dosages. That is to say that the effect provided by the additional agent of the present invention is greater than would be anticipated from the additive effects of the first agent and the additional agent when used separately. In an alternative embodiment, the first agent of the present invention and the additional agent are synergistic with respect to their side effects. That is to say that the side-effects caused by the additional agent in combination with the first agent are less than would be anticipated when the equivalent therapeutic effect is provided by either the first agent or additional agent when used separately.

It is expected that during the life of a patent maturing from this application many relevant 5HT2C receptor agonists and antagonists will be developed and the scope of the term 5HT2C receptor agonist and 5HT2C receptor antagonist is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### Examples

Reference is now made to the following examples, which together with the above descriptions illustrate some embodiments of the invention in a non limiting fashion.

### Materials and Methods

Mice: Adult (8-12 weeks of age; 20-25 g) males and females, SERT-Cre mice (B6.FVB(Cg)-Tg(Slc6a4-cre)ET33Gsat/Mmucd, (MMRRC, stock #031028-UCD), were used for the chemogenetic experiments. Adult C57Bl/6JOlaHsd mice (Envigo) and BALB/cOlaHsd (Envigo) were used for the characterization of the immune cells in the pineal gland, agonist, and social isolation experiments. Mice were maintained under specific-pathogen-free (SPF) conditions on a 12 h light:12 h dark cycle (lights on at 07:00) with food and water ad libitum. All experiments were performed in accordance with the National Institutes of Health Guidelines for the Care and Use of Laboratory Animals (Garber et al., 2011). All procedures and protocols were approved by the Technion Administrative Panel of Laboratory Animal Care. Throughout the experiments, mice were randomly assigned to experimental groups.

Stereotactic injections: Mice were anesthetized with a ketamine-xylazine mixture (ketamine 80 mg per kg body weight (mg/kg); xylazine 15-20 mg/kg; Sigma-Aldrich) in sterile saline (0.9% NaCl). After skull exposure, mice were fixed in a stereotactic frame (Stoelting) and the skull was carefully drilled, just above the target injection site. The needle was slowly lowered into the brain and left in place for 5 minutes, both before and after the injection. An adeno-associated virus 8 (AAV8)-based construct (ELSC Vector Core Facility) was used to induce Cre-dependent Gq-DREADD expression (AAV8-hSyn-DIO-hM3D(Gq)-mCherry), administered by injection of 1 ml (0.175 ml per minute) viral solution into DR (AP −4.36, ML ±0.2, DV 3.4; relative to bregma). Control mice were injected with a sham AAV8-hSyn-DIO-mCherry construct, lacking the DREADD gene. Mice showing signs of physical distress and pain were excluded from the experiment. Experiments were performed at least 21 days after surgery to ensure the expression of DREADDs and to allow full recovery after virus injection. Stereotactic injection sites were verified using fluorescence microscopy on brain tissue cryosections.

Chemogenetic neuronal manipulation: To activate DREADD-expressing neurons in the DR (Gq), all mice were i.p. injected with CNO (1 mg/kg; Sigma-Aldrich) in sterile saline twice a day for 3 days, or a single i.p. injection and brain was taken after 90 minutes for further analysis. Of note, in all experiments, mice in all groups, including the sham-injected controls, were injected with CNO, to control for any potential effects of CNO treatment.

Flow cytometry and CyTOF mass cytometer: Mice were euthanized and transcardially perfused with 20 mL PBS (without Ca²⁺/Mg²⁺). The brains were separated from the meninges and dissected. Non-neuronal cells were isolated by centrifugation through a 70-30% Percoll (Sigma-Aldrich, GE Healthcare Bio-Sciences) gradient to minimize effects on cell-surface proteins. Following extraction, brains were transferred to RPMI-1640 (Sigma-Aldrich) and dissociated using a Dounce homogenizer. The dissociated cell suspension, containing 30% Percoll, was then layered carefully on top of a 70% Percoll solution in PBS−/−. Following centrifugation (30 min at 500 g, 18° C., with minimal brake), the cells at the 70-30% interphase were removed, washed once with 1×PBS−/−, and filtered (40 mm strainer). The cell pellet was resuspended in 1 mL of staining buffer (PBS−/−, containing 1% bovine serum albumin and 0.05% sodium azide) and washed one more time.

For viability staining, cells (10⁶) were washed once with PBS, resuspended in Zombie NIR™ dye solution (1:1000, Biolegend, 423106) and incubated in the dark for 15 min at RT. Fc-blocking receptor (101320 Biolegend) was added for 15 minutes at RT. For extracellular staining, cells were washed with FACS staining buffer (PBS containing 1% bovine serum albumin, 1 mM EDTA and 0.05% sodium azide) and incubated with antibodies for 30 min at 4° C. The following mAbs were used: CD4-FITC GK1.5 (BioLegend 100406), CD8a-PerCP 53-6.7 (BioLegend 100732), CD19-APC 6D5 (BioLegend 115512), CD45-AF-700, 30F11 (BioLegend, 103128), MHCII-PB, M5/114.15.2, (BioLegend 107620), CD44-BV510 IMF (BioLegend 103044), CX3CR1-BV605 SA011F11 (BioLegend 149027), CD11b-PE M1/70 (Biolegend, 132704), TCRb-PE-CY5 H57-597 (BioLegend 109210), and CD11c-PE/Cy7 N418 (Biolegend, 117318). After incubation of the samples with the antibodies, the samples were washed twice with FACS staining buffer and re-suspended in 100 μl of 0.5% paraformaldehyde (PFA). Samples were analyzed using a CytoFLEX cell analyzer and FlowJo software. For CyTOF procedure, metal-conjugated antibodies (supplementary Excel 2) were incubated with cell lysate from brains of Control vs. Gq, without the meninges, after counting with trypan blue.

Tissue preparation and immunofluorescence: For validation of the virus injection site, evaluation of DREADDs, c-Fos expression, analysis of immune cells in the brain, and validation of flow cytometry results, brains were stained for immunofluorescence and visualized using fluorescence microscopy. Mice were euthanized and transcardially perfused with 20 mL PBS−/−. The scalp was removed and the skull was exposed. The skull was either detached or left intact, to include the pineal gland. Brains were either extracted and fixed in 4% PFA for 48 h, then cryoprotected in 30% sucrose solution (72 h), or snap-frozen over liquid nitrogen and fixed in methanol:acetone (1:1) for 10 min at −20⁰ C. Sagittal or coronal cryosections of the brains with or without the skull were sliced at 12 μm thickness. Sections were then mounted on super-frost slides (Thermo scientific).

For meninges and PG extraction, the intact brain with the skull was placed on a cold aluminum plate for 5 min to face the cold plate. The scalp was removed by two incisions from the occipital lobe to the squamosal sides, and towards the frontonasal suture. Then, the skull was carefully peeled off using microfine tweezers. A binocular microscope was used during the whole procedure to validate that the PG was removed along with the meninges. The meninges and PG were then placed and spread on pre-cooled superfrost slides and fixed with methanol:acetone (1:1) for 10 min in −20⁰ C. The slides were incubated in blocking buffer (1% bovine serum albumin in PBS−/− with 0.05% tween) for 1 hour at room temperature. Primary antibodies in blocking buffer were added, and incubated for 1 hour at room temperature. The following antibodies and concentrations were used, as summarized in Table 4:

TABLE 4

| Name | Catalog no. # | Company | Clone | Source | Dilution |
|---|---|---|---|---|---|
| CD45 | 103102 | Biolegend | 30F11 | rat | 1:250 |
| PECAM | 557355 | BD bioscience | MEC13.3 | rat | 1:250 |
| GFAP | Z0334 | DAKO | | rabbit | 1:500 |
| mCherry | Ab205402/7670-5 | Abcam/Encor | | chicken | 1:1000 |
| cFos | Ab190289 | Abcam | | rabbit | 1:1000 |
| Lyve-1 | 53-0443-82 | eBioscience | ALY7 | rat | 1:250 |
| CD11b | 101228 | Biolegend | M1/70 | rat | 1:250 |
| 5-HT_{2C} receptor | SC17797 | Santa Cruz | | mouse | 1:250 |

The slides were then washed and further incubated for 1 hour at room temperature with fluorescent-conjugated secondary antibodies, depending on the host of the primary antibody. The following secondary antibodies were used: Alexa Fluor 488-donkey anti-mouse IgG (1:500, Jackson ImmunoResearch laboratories, 715-545-151); Cy5 goat anti-rabbit IgG (1:500, Jackson ImmunoResearch laboratories, 111-175-144); Cy5 goat anti-rat IgG (1:500, Jackson ImmunoResearch laboratories, 112-175-167); Alexa-Fluor-568 goat anti-chicken IgY (1:500, Invitrogen, A11041); Alexa-Fluor-488 donkey anti-chicken IgG (1:500, Jackson ImmunoResearch laboratories, 703-545-155); and Alexa-Fluor-568 goat anti-rat IgG (1:500, Invitrogen, A11077). Slides were then washed three times in PBST (PBS−/− with tween 0.05%), mounted with DAPI-mounting medium (SouthernBiotech), and covered with a glass cover slip (Bar-Naor). All images were acquired using Zeiss Axio imager M2 microscope (Carl Zeiss Inc. US) or Zeiss Axio Imager.Z2 upright microscope. The quantification of positive or double-positive cells was performed using ImageJ (fiji) software or Zen image software. Image quantifications were analyzed using coronal and sagittal sections.

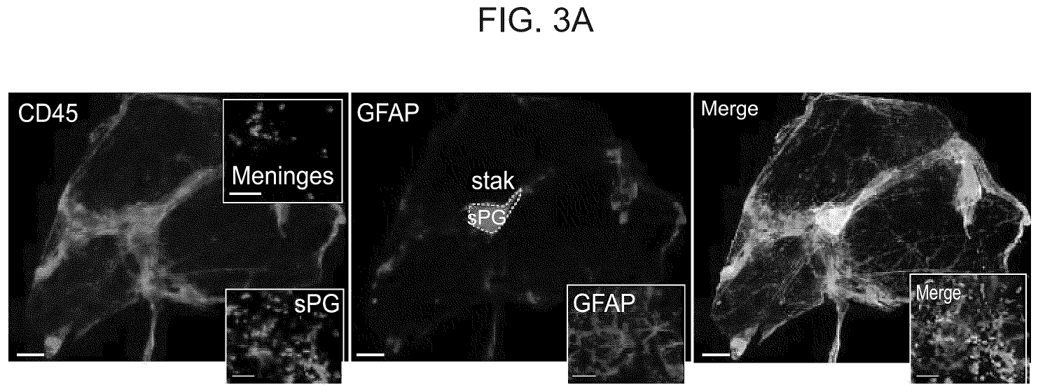

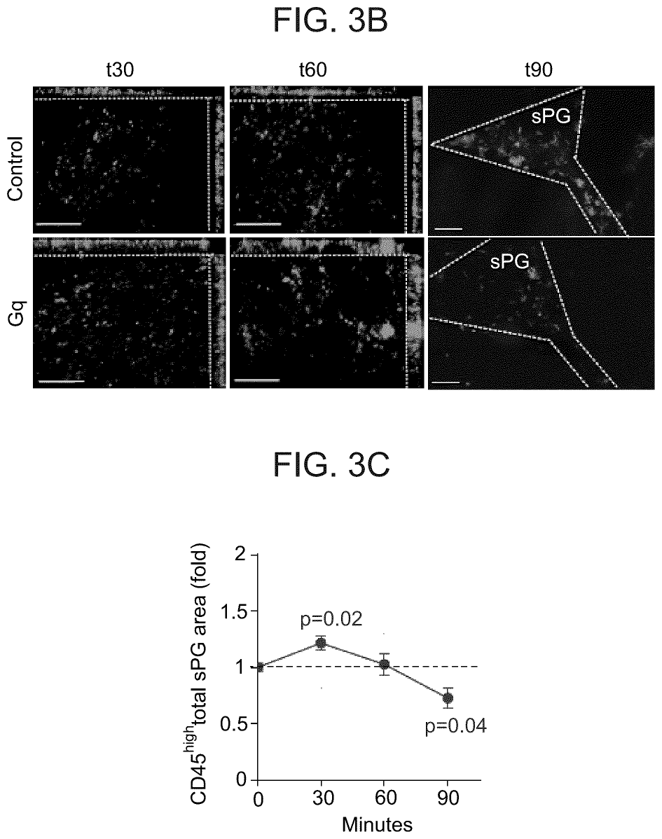

To visualize the pineal gland attached to the skull in FIG. 3A, Evans blue 2% was injected intraperitoneally for 2 hours.

Chemicals: CNO—(1 mg/kg; Sigma-Aldrich), Fluoxetine HCl (FLX)—(10 mg/ml; F0253000 sigma), 5-HT_{2B} Agonists (BW723C86, Cayman Chemicals), 5-HT_{2C} Agonist (ORG12962 Cayman Chemicals 27675), 5-HT_{2C} Antagonist (SB242084 Cayman chemicals 10096).

Depression experiments and behavioral analysis: To induce depression-like behavior, C57Bl/6 mice were socially isolated (SI) after weaning (4 weeks) and transferred to a clean single housing cage for 8 weeks. Control mice were housed in groups of 5. Validation of depression-like behavior induced by the 8-week isolation was done by the tail suspension test (TST). Next, saline, FLX, with or without antagonist 5-HT2C receptor was i.p. injected once a day for two weeks for the SI mice. Control mice were injected with saline.

Tail suspension test (TST) Mice were acclimated to the testing room for at least 1 hour before the behavioral experiment. Mice were hung by their tail in a designated apparatus for 6 min and recorded. A blinded experimentalist that does not know the identity of the experimental groups performed the analysis.

Single-cell preparation: Single-cell transcriptomic analysis was used to characterize the subsets of immune cells in the PG. After perfusion with PBS, the glands were mechanically extracted (pooled from 8 mice), and the tissue was dissociated to a single-cell suspension using a needle with decreasing sizes from 23 G−, followed by 25 G−, 27 G−. Large tissue fragments and cell aggregates were removed using a 70 mm cell strainer (Biologix 15-1070), centrifuges, and dead cells were manually counted using methylene blue (<10%).

Library prep and data generation: One RNA single cell library was prepared according to the 10× manufacturer's protocol (Chromium Next GEM Single Cell 3′ Library & Gel Bead Kit v3.1, PN-1000268) using 20,000 input cells. Single cell separation was performed using the Chromium Next GEM Chip G Single Cell Kit (PN-1000120). After construction, the concentration of the library was measured using Qubit (Invitrogen), and the size was determined using the TapeStation 4200 with the High Sensitivity D1000 kit (cat no. 5067-5584). RNAseq data was generated on Illumina NextSeq2000, P2 100 cycles (R1—28 bp, R2—90 bp, I1—10 bp, I2—10 bp) (Illumina, cat no. 20046811). The NGS run resulted in ˜530M reads. Primary analysis was performed using 10× Genomics software—Cell Ranger-V5.0.1 ((www(dot)10×genomics(dot)com/). We used the mouse genome reference provided by 10× genomics. After processing the raw data with CellRanger pipeline, 18,660 cells were obtained.

Data analysis: The obtained data was analyzed using the Seurat package. Low-quality cells with less than 600 or more than 20,000 UMIs, or more than 4000 genes, or with a frequency of mitochondrial genes above 20%, as well as genes detected in less than three cells were removed. After an initial round of analysis, a distinct erythrocyte cluster was removed as well as two additional clusters that were strongly defined by hemoglobin expression in differential expression analysis and considered as low quality. The cleaned dataset used for further analysis remained with 5156 cells. Next, the data was normalized by SCtransform (v2). The top 3000 variable genes of SCT normalization were used as input for Principal Component Analysis (PCA), from which the first 16 PCs were selected by the elbow heuristic for shared nearest-neighbor clustering (Louvain algorithm). Clustering resolution was set to 0.3, resulting in 14 clusters, and was visualized by Uniform Manifold Approximation and Projection (UMAP). Two overlapping clusters characterized by similar signature, were combined to one single cluster (“Cluster1”, FIG. 3A), giving rise to 13 clusters. We then annotated the cell types of each of the 13 clusters manually based on cluster-specific markers from differentially expression analysis (Wilcoxon rank sum test on log normalized data) and known cell-type gene expression marker from the literature and databases. Cluster1 myeloid cells were subsetted for subclustering analysis, resulting in 1845 cells. For clustering, the first 12 PCs from the top 3000 variable genes (after SCT normalization) were considered at a clustering resolution of 0.5, resulting in six distinct subclusters. Gene expression levels in Figures are presented as log-normalized data (scaling factor=1000) if not stated otherwise. The average heatmap was generated using the ComplexHeatmap package.

MERFISH: To prepare the tissue for MERFISH, the OCT-embedded tissue was placed for 30 min in −20° C. cryostat chamber before sectioning. 10 mm sagittal tissue of the desired area was loaded to a functionalized bead coated coverslip provided by Vizgen and fixated with 4% PFA, washed and placed in 70% ethanol until further use. The Gene panel for MERFISH was composed of 302 protein-coding genes including known neuronal, glial, vascular, immune cell type markers, immune and glial reactivity genes and neuronal receptors.

Hybridization: Before hybridizing the sample with the gene panel (=library mix), the sample was washed with 5 ml Vizgen Sample Prep Wash Buffer (Vizgen part number 20300001) and then incubated in 5 ml Formamide Wash Buffer (Vizgen pn 20300002) at 37° C. for 30 min in an incubator. The Formamide Wash Buffer was aspirated from the tissue, and 50 μl of the gene panel mix was added on top of each tissue. A piece of parafilm ˜1.5 cm×1.5 cm was placed on top to spread the library mix and protect it from evaporation. The dishes were sealed with parafilm and placed in a humidified incubator at 37° C. for 36-48 hours. The parafilm was removed from the top of each tissue, and the samples were incubated in 5 ml Formamide Wash Buffer at 47° C. for 30 minutes, twice. The samples were then washed with 5 ml Sample Prep Wash Buffer for 2 minutes.

Gel embedding: To gel embed the samples, fresh 10% w/v ammonium persulfate solution was prepared. For each sample, 5 ml of Gel Embedding Premix (Vizgen pn 20300004) was combined with 25 μl of the 10% ammonium persulfate solution and 2.5 μl of TEMED (N,N,N′,N′-tetramethylethylenediamine). In parallel, one 20 mm Gel Coverslip (Vizgen pn 20400003) for each sample was cleaned with RNAseZap, followed by 70% ethanol and dried with Kimwipes. The Gel Coverslips were then covered with 100 μl of Gel Slick Solution (VWR, catalog number 12001-812) for a minute and wiped dry with Kimwipes. The Sample Prep Wash Buffer was aspirated from the samples. For each sample, 100 μl of the Gel Embedding Mix was retained in a small tube, while the remainder of the Gel Embedding Mix was added to the samples and incubated for 1 minute. The Gel Embedding Mix was then poured out from the samples into a waste tube but kept aside on the bench (to monitor gel formation). The slides were then aspirated dry, leaving just enough liquid to keep the tissue from drying out. 85 μl of the separately retained Gel Embedding Mix was added on top of the tissue, and the Gel Slick treated coverslip was placed over it with tweezers, with the Gel Slick-treated side facing down toward the tissue and avoiding air bubbles. Extra Gel Embedding Solution was aspirated from the sides of the coverslips. The dishes were incubated at room temperature for 1.5 hours to allow the gels to form. Thereupon, the coverslips were removed using a Hobby Blade and tweezers.

Tissue clearing: To clear the samples of lipids and proteins that interfere with imaging, 5 ml of Clearing Premix (Vizgen pn 20300003) were mixed with 50 μl of Proteinase K for each sample. After the coverslips were removed from the gel embedded samples, the clearing solution was added to each sample, and the dishes were sealed with parafilm. The samples were placed at 47° C. in a humidified incubator overnight (or for a maximum of 24 hours), and then moved to 37° C. The samples were stored in the Clearing solution in the 37° C. incubator prior to imaging for up to a week.

Sample imaging: The Clearing solution was aspirated from the sample, and the sample was washed three times with Sample Prep Wash Buffer briefly, then again for 10 minutes on a rocker, and then three more times briefly. The sample was incubated with 3 ml of the appropriate hybridization buffer, including DAPI and polyT reagent (Vizgen pn 20300021), for 15 minutes at room temperature on a rocker, covered from light. The sample was then washed with 5 ml of the Formamide Wash Buffer (Vizgen pn 20300002) for 10 minutes at room temperature on a rocker, covered from light, and then transferred to 5 ml of the Sample Prep Wash Buffer (Vizgen pn 20300001). In the meantime, the Imaging buffer was prepared by combining the Imaging buffer, the Imaging Buffer Activator (Vizgen pn 20300015), and RNase inhibitor at a ratio of 500:2.5:1. The hybridization buffers appropriate to the gene panel, as well as the imaging buffers, were loaded onto the Vizgen microscope system. The sample was placed in the flow chamber and connected to the fluidics system of the Vizgen microscope, taking care to disperse air bubbles. A low-resolution mosaic was acquired using a 10× objective, and the regions of interest corresponding to the whole sagittal brain area were selected for high-resolution imaging with a 60× lens. For the high-resolution imaging, the focus was locked to the fiducial fluorescent beads on the coverslip. Seven 1.5 μm-thick z planes were taken for each field of view when imaging the tissue, including for the DAPI channel (nuclear staining) and polyT channel (staining for total mRNA). Images were decoded to RNA spots with xyz and gene ID using Vizgen's Merlin software.

### MERFISH Analysis

Segmentation: To obtain high-quality segmentations of single cells, we established a two-step segmentation procedure. First, Watershed algorithm using DAPI nuclear seeds and polyT total mRNA staining basins was used to obtain cellular masks. Next, these initial segmentations were provided as priors to the Baysor algorithm, together with all decoded transcript data. Baysor is a segmentation method for imaging-based transcriptomics that takes advantage of high-dimensional molecular information contained therein to segment cell boundaries by considering joint likelihood of transcriptional composition and cell morphology. Whole section MERFISH data were split into 8 large tiles and Baysor was run on each tile with the same parameters (-m=30, -s=6, --n-clusters=12, --prior-segmentation-confidence 0.15, --scale-std=40%), afterwards the tiles were merged back together for the single cell analysis of full section.

Analysis of spatial single cell data: After segmentation, spatial single cell gene expression data were analyzed in R using mainly Seurat package. Only cells that passed the following quality control criteria were considered for further analysis: between 10 and 3000 detected molecules, more than 5 detected genes, average confidence in transcript assignment higher than 0.85, area between 3 and 600 μm², transcript density less than 15 and elongation less than 50. Data were normalized and scaled using SCTransform function and principal component analysis (PCA) was then performed on all features. First 25 principal components were used for UMAP embedding and to calculate shared nearest-neighbor graph. Louvain algorithm with resolution 0.1 was then used for initial coarse-grained clustering, and marker genes were identified using Wilcoxon rank-sum test on log-normalized count values. Next, individual clusters corresponding to major cell types were iteratively subsetted and aforementioned workflow was repeated on these subsets to refine cellular annotations based on known marker genes from published literature, reference atlases and cellular location. Afterwards, cells were classified into major categories. For analysis of myeloid subsets, cells annotated as Immune cells were separated. The residual effect of contaminating RNA was then removed by regressing expression signatures of other, non-immune cell types from the data. For this, the inventors first obtained signatures of every non-immune cell type by i) searching for differentially expressed (DE) genes between Immune cluster and other coarse clusters using strict threshold and ii) intersecting these DE genes with identified markers of every cell type. They then calculated aggregate score for each signature in each single cell using Seurat's AddModuleScore function and regressed these scores by passing them as a variable to SCtransform before repeating PCA, UMAP, subclustering (using first 15 principal components), and differential expression analysis.

Statistical analysis: Fold change was used when describing the ratios between groups when experiments where pooled together in different timings. The significance levels of the data were determined using GraphPad PRISM version 9.0.0(121). Two-tailed unpaired Student's t-test analyzed experiments comparing two groups, and one-way ANOVA Multiple comparison tests (Bonferroni, specifically mentioned for every experiment) were used with a significance threshold set at P=0.05 or False Discovery Rate (FDR)=0.05. P values are presented in graphs. Data are represented as mean±standard error of the mean (s.e.m). Outliers were excluded using the ROUT method for outlier identification (Motulsky and Brown, 2006) with a False Discovery Rate less than 1% (Q<1%). All experiments were performed at least twice.

### Results

The stimulatory (hM3Dq; Gq) form of Designer Receptor Exclusively Activated by Designer Drugs (DREADDs) was used to induce DR serotonergic activation directly so as to determine whether serotonin can affect the infiltration of peripheral immune cells into the brain compartment. Upon exposure to a synthetic ligand, clozapine-N-oxide (CNO), this Gq receptor elicits an intracellular cascade that leads to neuronal activation. The Gq DREADD receptor was expressed under the Synapsin promoter in serotonergic neurons using an adeno-associated virus (AAV8)-based vector that was stereotactically injected into the DR. The genetic specificity to serotonergic neurons was achieved using transgenic mice that expressed Cre recombinase under the promoter of the serotonin transporter (SERT). SERT transports serotonin from the synaptic cleft to the presynaptic neuron and its corresponding gene Sl6ca4 is highly expressed in serotonergic neurons. To visualize the DREADD expression, the virus also carried a gene encoding for mCherry fluorescent reporter. As a control in this study, mice were injected with the same virus encoding the mCherry fluorescent reporter but lacking the DREADD gene (control virus). This group could control for any potential local inflammatory response induced by the viral infection, effects of surgery, and CNO administration.

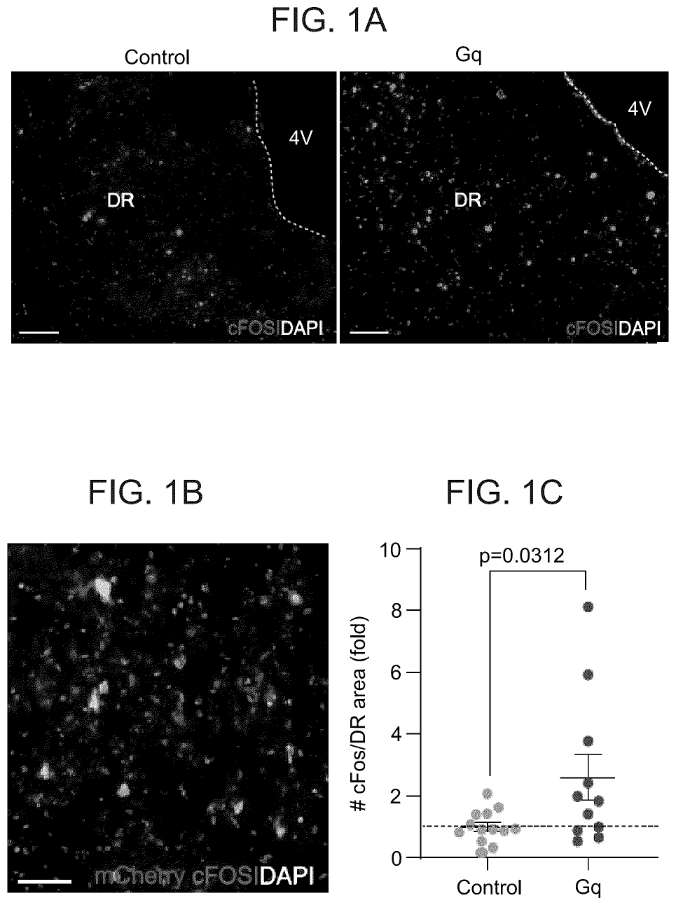

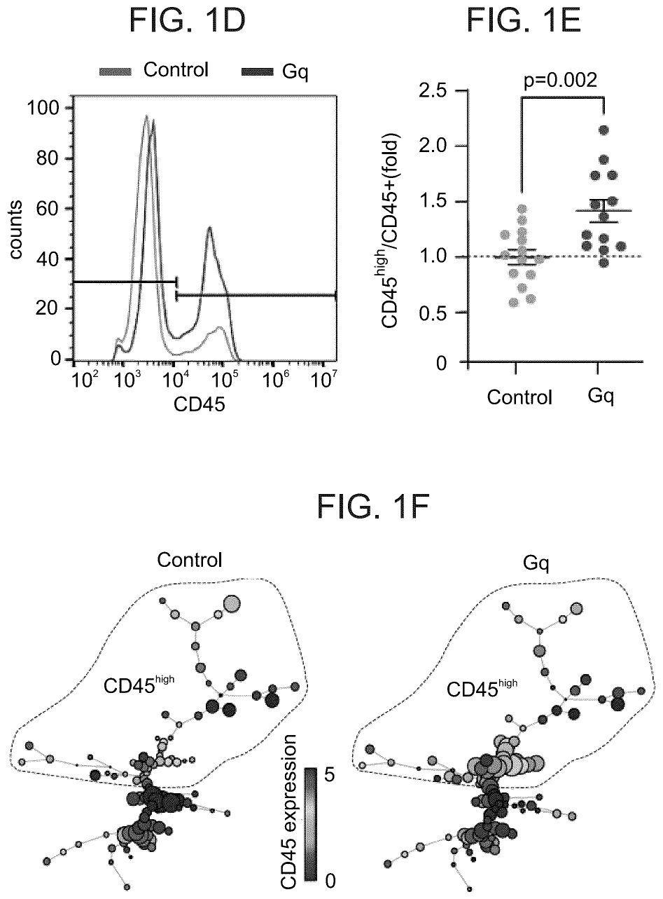

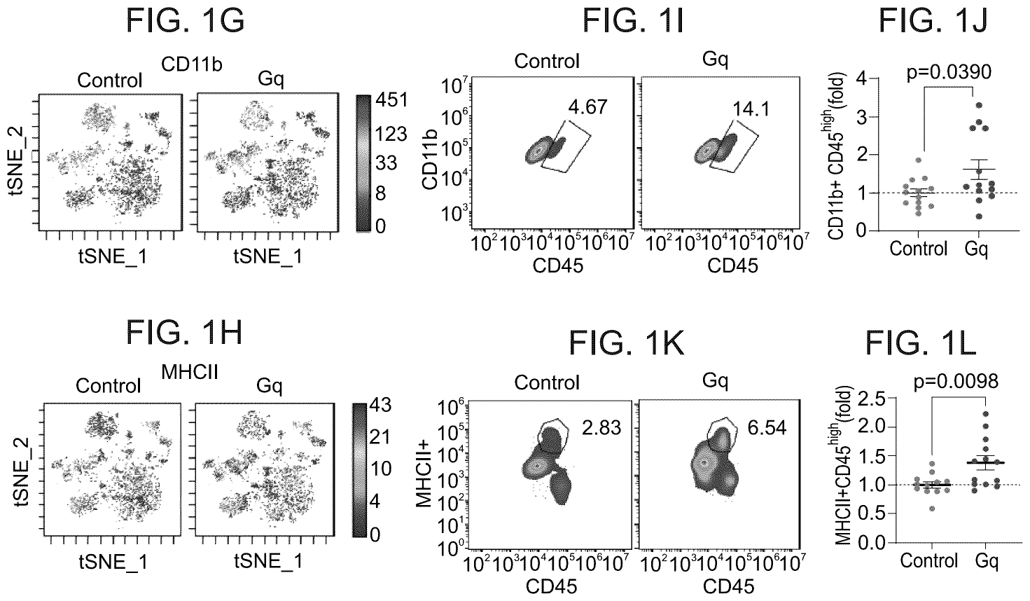

To validate the functional effects of the DREADD manipulation on neuronal activation, c-Fos levels were analyzed. This early activation marker is often used as a proxy of neuronal activation. It was found that following CNO injection, there was a 2.5-fold increase in c-Fos⁺ cells in the DR of the Gq group compared to the mice injected with the control virus (Control 1±0.1, Gq 2.5±0.7, t-test, p=0.0312, FIGS. 1A-C), indicating that the DREADD manipulation induced targeted neuronal activation in the DR.

To determine the functional effects of DR activation on the immune system, CNO was injected to the Gq-injected mice and their controls, and immunological changes were analyzed at two-time points reflective of acute and chronic serotonergic activation (“immediate”—90 min after CNO application, and “repeated”—after 3 days of repeated CNO applications). The immune cell populations were examined in the periphery (spleen, blood, bone marrow) and in the brain compartment (parenchyma including choroid plexuses; CP). In the brain compartment, the effects were evident even 90 minutes following a single DR serotonergic activation manifested by an increase in peripheral immune populations in Gq vs. control mice (Control 1±0.06 vs. Gq 1.45±0.1, t-test p=0.002, FIGS. 1D and E). The present inventors initially characterized this peripheral immune population by its high CD45 expression (CD45^{high}), which is much lower in steady-state resident microglia. Since the immediate time point (90 min) represents a more direct effect of the serotonergic manipulation and its functional outcomes, we focused on this time point, using a single CNO injection, for the remainder of the study.

To gain a broad perspective of the changes in the brain's immune compartment following DR activation, we used cytometry by time of flight (CyTOF). The main changes were seen in the infiltrating immune populations (FIG. 1F) identified by their expression of CD45^{high}/CD44⁺ (Korin et al., 2017) and CD45^{high}/CD43⁺ (Borst and Prinz, 2020). CyTOF analysis of infiltrating immune cells revealed that CD11b⁺ and MHC class II⁺ cells were elevated in Gq mice compared to control mice (FIGS. 1G and 1H). Flow cytometry further validated these results (control 1±0.1 and 1±0.05 vs Gq 1.6±0.2 and 1.38±0.1 t-test p=0.039 and p=0.009, respectively) (FIGS. 1I-L). Thus, chemogenetic serotonergic activation, induced an increase in peripheral immune cell abundance in the brain compartment.

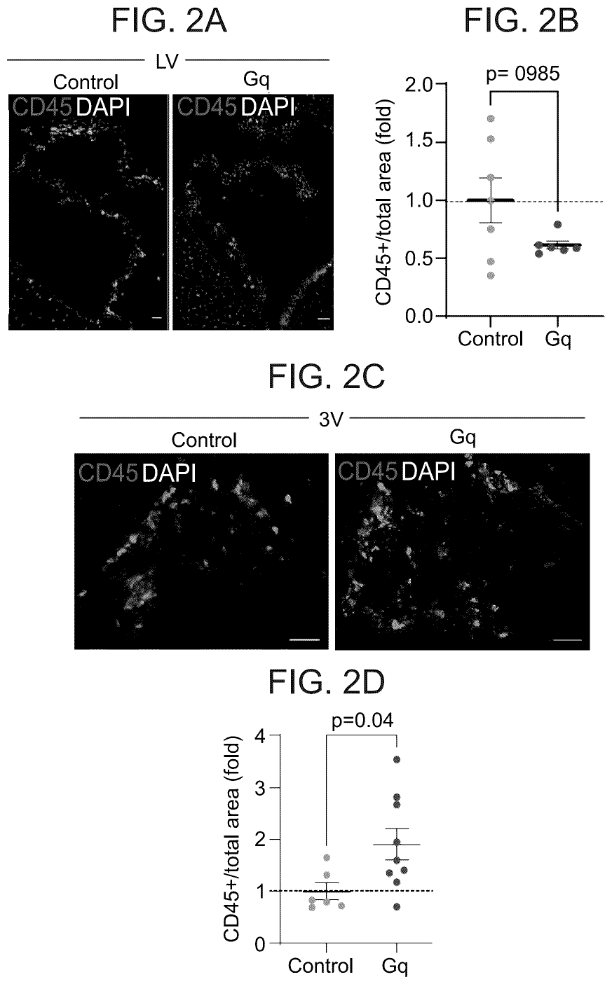

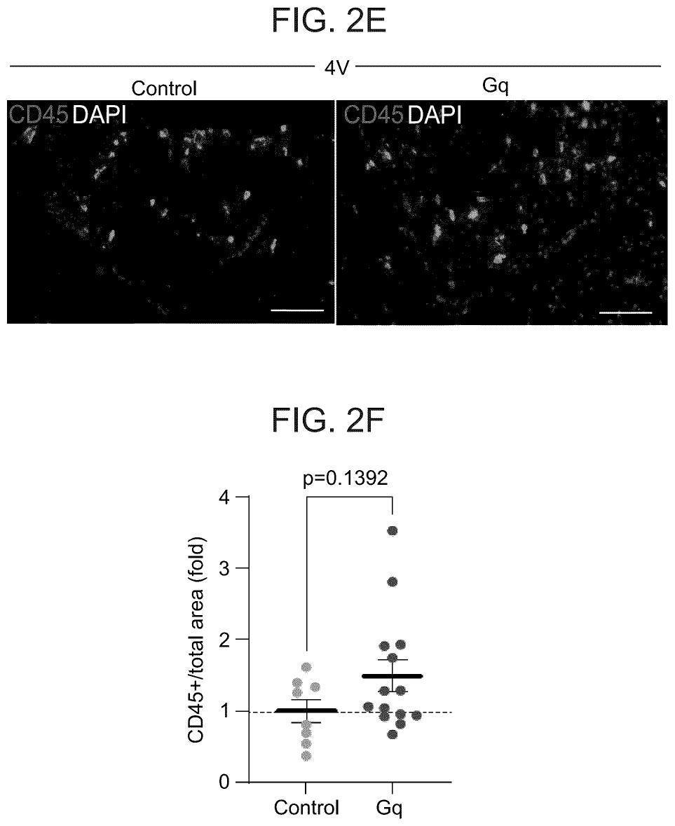

Such a rapid increase of immune populations is somewhat surprising and can reflect a brain-wide increase in infiltrating immune cell abundance or, alternatively, locally within a specific infiltration site. Therefore, the present inventors focused their analysis on characterizing CD45 expressing cells in the brain's borders, specifically the ventricles, known as immune infiltration sites (FIGS. 2A-F). Moreover, as mentioned earlier, DR serotonergic neurons were previously shown to project to the ependymal ventricle walls and CP. Interestingly, it was found that the most significant increase in the number of immune cells, was in the 3^{rd} ventricle (Control: 1+0.15 vs. Gq: 1.9+0.3, t-test p=0.04, FIGS. 2C and D). A similar yet non-significant trend was also evident in the 4^{th} ventricle (FIGS. 2E and F), but in the lateral ventricles, there was no difference in immune populations between the two groups (FIGS. 2A and B). Thus, the serotonergic manipulation had a local effect and increased the peripheral immune populations, mainly in the CP of the 3^{rd} ventricle.

The 3^{rd} ventricle is anatomically unique as it is connected directly to the pineal gland (PG), an endocrine gland. This gland is mainly known for its melatonin production by pinealocytes, non-classical neurons. In addition to pinealocytes, the PG is comprised of glia, endothelial, epithelial, and immune. The presence of immune cells in this site is not surprising since the PG is a major secretory circumventricular organ (CVO) and, thus, characterized by permeable capillaries. The PG is further connected to the 3^{rd} ventricle via its stalk, thereby forming the deep pineal gland (dPG), which is attached to the ependymal wall of the 3^{rd} ventricle roof, between the habenular and the posterior commissure. Attached to the dPG, the pineal recess (Pr) emerges, which is the original recess of the 3^{rd} ventricle.

Anatomically, the PG is located between the meninges and the 3^{rd} ventricle and innervated by serotonergic inputs. Functionally, the increase in immune cell abundance in response to serotonergic stimulation was localized to the 3^{rd} ventricle. This collective evidence has led the present inventors to hypothesize that the PG acts as a potential conduit for immune cells mobilization into the ventricle walls in response to serotonergic activation. Since the PG is connected to the meninges, the PG was extracted along with the meninges and stained for CD45 to visualize immune cells, and for GFAP, expressed on astrocytes, and thus, labels a parenchymal tissue (this staining was also validated in BALB/c mice) (FIG. 3A). The PG is considered parenchyma and is known to be populated by astrocytes. Although the immune cells appear to reside both in the meninges and the PG continuously, only the PG is populated with GFAP-positive cells alongside immune cells. As the meninges, the sPG is embedded with blood and lymph vessels (vasculature marker, CD31, and the lymphatic marker, LYVE1). However, in the PG stalk and Pr only blood vessels were detected. This finding is in agreement with the definition of the PG as a CVO that has increased accessibility to the periphery.

Next, the DREADD-induced activation of the DR serotonergic neurons was repeated and the distribution of immune cells in the PG at various time points following the CNO application was analyzed. Initially, 30 min post-CNO application (t30), a significantly increased abundance of CD45+ cells was found in the sPG. However, 60 and 90 minutes (t60 and t90) following the serotonergic activation, CD45⁺ cells decreased in abundance compared to the control (FIGS. 3B and 3C). These findings suggest that the PG is involved in immune cell mobilization to the 3^{rd} ventricles following serotonergic activation.

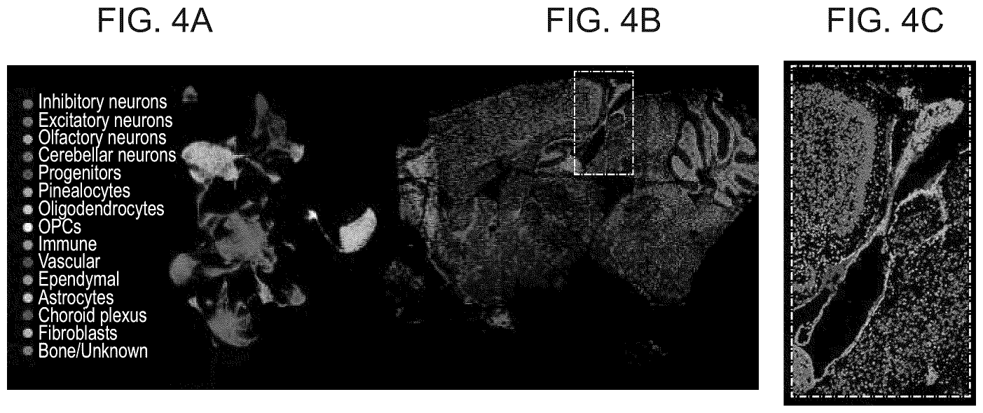

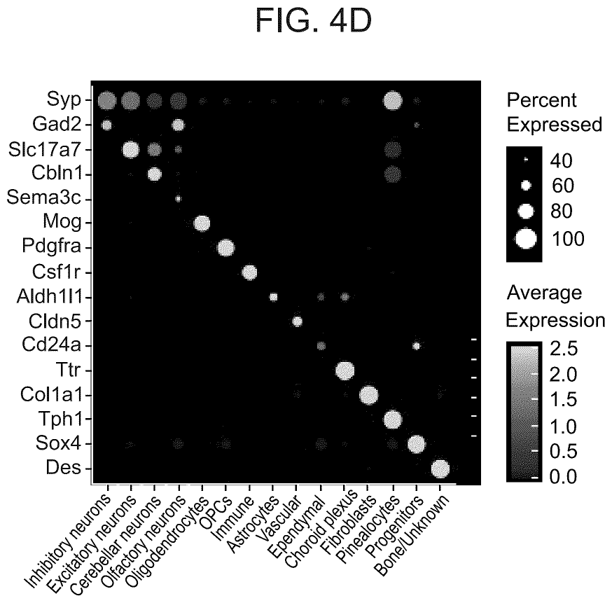

To further understand the anatomical and functional role of the PG in the potential infiltration of immune cells, spatial transcriptomic analysis was used. Specifically, the present inventors used multiplexed error-robust FISH (MERFISH (Chen et al., 2015)), an imaging-based, single cell-resolved spatial transcriptomic technology, to measure a panel of 302 genes in a mid-sagittal section that included the skull to keep the PG intact. After segmenting MERFISH data into single cells, the analysis pipeline (described in methods) identified 103516 cells annotated into 15 major cell populations (including neuronal, glial, immune, vascular, and other cell types), which allowed them to map each cell to its original location in the tissue (FIGS. 4A and B). PG structure was clearly visible in the area above the third ventricle and characterized by the abundance of pinealocytes (marked by the expression of Tph1 and Asmt), validating the experimental and computational approach (FIGS. 4C and D). The present inventors then performed clustering analysis of the myeloid cells, which identified a cluster of resident myeloid cells, microglia (marked by Selplg, Tmem119) and non resident myeloid cells (marked by Mrc1, Cd163). Interestingly, they found that an additional third cluster of myeloid cells, characterized by high expression levels of Cd74, an MHCII-associated invariant chain, were uniquely localized to the PG. To further characterize this population, they performed single-cell RNA sequencing (scRNA-seq) in a new set of mice. Together, the MERFISH and scRNA-Seq analysis identified a uniquely-localized Cd74 and MHCII+-enriched myeloid population in the PG. Based on this evidence, the present inventors suggest that the PG comprises a specialized immune environment within the brain compartment, potentially positioned for antigen presentation and controlled by the serotonergic system.

To understand how this environment could be affected by serotonergic activity, the present inventors examined the expression of the serotonergic receptors expressed in the PG. Based on our MERFISH and scRNA-Seq data, one serotonin receptor, Htr2c, was specifically enriched in the PG area and CP (FIG. 5A). Further analysis revealed that it is enriched in a specific cluster (cluster 10) (FIG. 5B). This is a cluster of CP cells that share many similarities with ependymal cells, a diverse cell population located in the lining of the brain ventricles. The main role of ependymal cells is to maintain cerebral spinal fluid circulation, but they were also shown to facilitate immune cell trafficking to the ventricles and parenchyma. The present MERFISH analysis localized Htr2c expressing cells mainly in the choroid plexus cells and some in the attached recess of the PG (FIG. 5C). Using immunohistochemistry (IHC), the present inventors validated 5-HT_{2C} receptor expression and found this receptor to be expressed in the PG area, and the extended choroid plexus of the 3^{rd} ventricle (FIG. 5D). This evidence suggests that the 5-HT_{2C} receptor is a potential candidate to mediate the serotonin-induced immune cell trafficking to the brain compartment.

To directly test the involvement of the 5-HT_{2C} receptor in immune cell trafficking, mice were treated with ORG12962, a 5-HT_{2c} agonist. As a control, an agonist specific for 5-HT_{2B} receptor (BW723C86) was used, which has a very limited expression in the brain (Acevedo-Triana et al., 2017), and as an additional control, saline was used. Within two hours of i.p. injection of the 5-HT_{2C} receptor agonist, immune cell abundance was increased in the brain compartment (FIGS. 6A and B), validating the capacity of this serotonergic receptor to regulate peripheral immune cell trafficking.

These findings raised a potential clinical relevance since one of the most important aspects of serotonin is its involvement in depression. Pharmacologically, serotonin-modulating drugs, specifically, selective serotonin reuptake inhibitors (SSRIs), are prevalently used for treating depression. Increasing evidence suggest that immune mediators may be linked to the emergence and treatment of depression, and studies report changes in microglia and peripheral immune cells during depressive episodes. However, it remains unknown whether the infiltrating immune populations play a role in depression or the therapeutic effect of SSRIs.

To correlate the present findings to the clinical aspect, the present inventors analyzed the abundance of peripheral immune cells in the brains of socially isolated (SI) mice, a common model of depression (FIG. 6C). Based on the findings that serotonin is required for peripheral immune infiltration to the brain compartment, a reduction in the abundance of peripheral cells in the brain compartment of SI mice was expected as depression is associated with an impairment in serotonin levels. Indeed, SI mice manifested decreased abundance of CD45^{high} cells (control group-housed 1±0.07 vs. SI saline 0.67±0.1) in their brain compartment (FIGS. 6D and E).

SSRIs increase the levels of available serotonin by inhibiting serotonin reuptake from the synapse. Thus, based on the present findings, SSRIs should increase the immune cell abundance in the brain of the treated mice. SI mice were treated with an SSRI, fluoxetine (FLX; i.p, 10 mg/kg) for 14 days. As expected, SSRI treatment increased the abundance of peripheral immune cells compared to non-treated SI mice (SI FLX 1±0.06, FIGS. 6D and E), suggesting that the SSRI function mechanism involves peripheral immune infiltration. To determine whether this effect is dependent on the 5-HT_{2C} receptor, a relevant antagonist (SB242084) was used. Application of this antagonist to SI mice attenuated the SSRI-induced increase in the abundance of CD45^{high} cells (control group-housed—1±0.07, SI saline—0.6±0.1, SI FLX—1±0.06, SI FLX+5-HT_{2c} antagonist—0.6±0.04, p=0.0012, FIGS. 6D and E). Thus, SSRI treatment induces peripheral immune cell infiltration to the brain compartment, and this effect requires a functional 5-HT_{2C} receptor.

Finally, to determine whether these findings were also relevant to the clinical effects of SSRI, the present inventors monitored the behavior of SI mice, using the tail suspension test (TST). This paradigm is based on the notion that in depression mice models, when mice are suspended by their tail, they manifest an immobile posture as a behavioral trait associated with depression. Various antidepressant medications reverse immobility and promote the occurrence of escape-related behavior. An increase in immobility time in the SI mice was observed as compared to group-housed controls. This immobility time was decreased in the SSRI-treated group. However, this beneficial effect of the SSRI was attenuated when the SI mice were also treated with the 5-HT_{2c} receptor antagonist together with the SSRI (control group-housed mice—1±0.1, SI saline—1.67±0.09, SI FLX—0.99±0.2, SI FLX+5-HT_{2c} antagonist—1.3±0.3, ANOVA p=0.0005, FIG. 6F). Thus, although serotonin has at least seven types of receptors in the brain (5-HT1-7), inhibition of the 5-HT_{2C} receptor was sufficient to prevent peripheral immune infiltration to the brain compartment and to abrogate the beneficial behavioral effects of the SSRI treatment.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting.

In addition, any priority document(s) of this application is/are hereby incorporated herein by reference in its/their entirety.

What is claimed is:

1. A method of treating a disease or disorder for which an increase in immune cells in the brain is therapeutic in a subject in need thereof, comprising administering to the subject a therapeutically effective amount of an agent, wherein said agent:
(i) specifically binds and increases activity of a receptor selected from the group consisting of a 5HT2C receptor, a 5HT2A receptor and a 5HT2B receptor in the postcentral gyrus and/or the choroid plexus (CP) of the brain of the subject; and/or
(ii) increases the amount of serotonin in the brain of the subject,
thereby treating the disease, with the proviso that the disease is not depression or anxiety.

2. The method of claim 1, wherein said agent specifically binds and increases activity of a receptor selected from the group consisting of a 5HT2C receptor, a 5HT2A receptor and a 5HT2B receptor in the postcentral gyrus and/or the choroid plexus (CP) of the brain of the subject.

3. The method of claim 2, wherein said receptor is a 5HT2C receptor.

4. The method of claim 1, wherein said disease is a neurodegenerative disease or a mood disorder.

5. The method of claim 1, wherein said disease is selected from the group consisting of schizophrenia, post-traumatic stress disorder (PTSD), epilepsy, autism, stroke, ischemia, Alzheimer's disease, Parkinson's disease, migraine and cancer.

6. The method of claim 3, wherein said agent is selected from the group consisting of Lorcaserin, Vabisacerin, RO 60-01752, YM-348, mCPP, CP809101, WAY163909 and ORG12962.

7. The method of claim 3, wherein said agent is an activating antibody.

8. The method of claim 3, further comprising administering to the subject an additional agent which specifically targets and activates a receptor selected from the group consisting of 5HT1B, 5HT2A and 5HT2B.

9. The method of claim 1, further comprising administering to the subject an additional agent selected from the group consisting of a selective serotonin reuptake inhibitor (SSRI), a serotonin and norepinephrine reuptake inhibitor (SNRI), bupropion, a tricyclic antidepressant and a monoamine oxidase inhibitor (MAOI).

10. The method of claim 1, wherein said agent increases the amount of serotonin in the brain of the subject thereby treating the disease.

11. The method of claim 10, wherein said agent is selected from the group consisting of a selective serotonin reuptake inhibitor (SSRI), a serotonin and norepinephrine reuptake inhibitor (SNRI), bupropion, a tricyclic antidepressant and a monoamine oxidase inhibitor (MAOI).

12. A method of treating a disease or disorder for which a decrease in immune cells in the brain is therapeutic in a subject in need thereof, comprising administering to the subject a therapeutically effective amount of an agent which specifically binds and reduces activity of a receptor selected from the group consisting of a 5HT2C receptor, a 5HT2A receptor and a 5HT2B receptor in the postcentral gyrus and/or the choroid plexus (CP) of the brain of the subject, thereby treating the disease.

13. The method of claim 12, wherein the disease is an autoimmune disease.

14. The method of claim 12, wherein said agent is an antibody.

15. The method of claim 12, wherein said receptor is a 5HT2C receptor.

16. The method of claim 15, wherein said agent is a 5HT2C antagonist.

17. The method of claim 12, further comprising administering to the subject a therapeutically effective amount of an additional agent selected from the group consisting of a selective serotonin reuptake inhibitor (SSRI), a serotonin and norepinephrine reuptake inhibitor (SNRI), bupropion, a tricyclic antidepressant and a monoamine oxidase inhibitor (MAOI).

18. An article of manufacture comprising a first agent which specifically binds and increases activity of a 5HT2C receptor; and at least one additional agent selected from the group consisting of a selective serotonin reuptake inhibitor (SSRI), a serotonin and norepinephrine reuptake inhibitor (SNRI), bupropion, a tricyclic antidepressant, a monoamine oxidase inhibitor (MAOI) and an agent which specifically targets and activates a receptor selected from the group consisting of 5HT1B, 5HT2A and 5HT2B.

19. The article of manufacture of claim 18, wherein said first agent is selected from the group consisting of Lorcaserin, Vabisacerin, RO 60-01752, YM-348, mCPP, CP809101, WAY163909 and ORG12962.

20. The article of manufacture of claim 18, wherein said first agent is an activating antibody.
